(19) United States
(12) Patent Application Publication
(10) Pub. No.: US 2018/0092951 A1
(43) Pub. Date: Apr. 5, 2018

(54) VIROTHERAPY WITH AN ANTIBODY COMBINATION

(71) Applicant: STEMINNUNE, INCORPORATED, San Diego, CA (US)

(72) Inventors: Aladar SZALAY, Highland, CA (US); Boris MINEV, San Diego, CA (US)

(73) Assignee: StemImmune, Incorporated, San Diego, CA (US)

(21) Appl. No.: 15/559,028

(22) PCT Filed: Mar. 17, 2016
(86) PCT No.: PCT/US16/22978

§ 371 Date: Sep. 15, 2017

Related U.S. Application Data
(60) Provisional application No. 62/135,096, filed on Mar. 18, 2015.

(51) Int. Cl. A61K 35/768; C07K 16/22; C12N 15/863; C07K 16/28; C07K 16/30; A61K 9/00; A61P 35/00

(52) U.S. Cl. CPC A61K 35/768; C07K 16/22; C12N 15/8636; A61K 2039/507; C07K 16/30; A61K 9/0019; A61P 35/00; C07K 16/2863

(57) ABSTRACT

Disclosed herein are viruses that can be used in methods of treatment for cancer. More specifically, the viruses express two or more antibodies which induce an effective anti-tumor immune response. The viruses also can be used in diagnostic methods.

### CROSS-REFERENCE TO RELATED APPLICATIONS

This application claims the benefit of and priority to U.S. Provisional Application No. 62/135,096, filed Mar. 18, 2015, the entire contents of which are hereby incorporated by reference.

### BACKGROUND OF THE INVENTION

Cancer is the second most common cause of death in the United States, exceeded only by heart disease. In the United States, cancer accounts for 1 of every 4 deaths. The 5-year relative survival rate for all cancer patients diagnosed in 1996-2003 is 66%, up from 50% in 1975-1977 (Cancer Facts & Figures American Cancer Society: Atlanta, Ga. (2008)). Discovering highly effective cancer treatments is a primary goal of cancer research.

### SUMMARY OF THE INVENTION

The present invention relates generally to the treatment of human cancer and, more specifically, to use of several treatment modalities in combination to induce effective anti-tumor immune responses.

Disclosed herein, in some embodiments, is a method for treating a solid tumor, comprising administering to the subject a recombinant virus that can infect a cell in the tumor, or a cell comprising a virus that can infect a cell in the tumor, wherein the virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein targeting the TME. In some embodiments, the virus is an oncolytic virus. In some embodiments, the oncolytic virus is a vaccinia virus. In some embodiments, the virus is a replication-competent oncolytic vaccinia virus (VACV). In some embodiments, the virus expresses two or more antibodies targeting the TME. In some embodiments, the two or more antibodies are selected from the group consisting of: (i) an antibody that binds to a protein that stimulates angiogenesis and/or vascularization, (ii) an antibody that binds to a receptor tyrosine kinase selected from the group consisting of epidermal growth factor receptor (EGFR), Her2/c-neu, Her3 and Her4, and (iii) an antibody that binds to a protein involved in the development of epithelial-mesenchymal interactions. In some embodiments, the two or more antibodies are selected from the group consisting of: an antibody that binds to vascular endothelial growth factor (VEGF), an antibody that binds to epidermal growth factor receptor (EGFR), and an antibody that binds to fibroblast activation protein (FAP). In some embodiments, one of the two or more antibodies is an antibody that binds to VEGF. In some embodiments, the antibody that binds to VEGF is G6-31. In some embodiments, one of the two or more antibodies is an antibody that binds to EGFR. In some embodiments, the antibody that binds to EGFR is anti-EGFRVHH. In some embodiments, one of the two or more antibodies is an antibody that binds to FAP. In some embodiments, the antibody that binds to FAP is M036. In some embodiments, the two or more antibodies comprise an antibody that binds to VEGF and an antibody that binds to EGFR. In some embodiments, the two or more antibodies comprise an antibody that binds to VEGF and an antibody that binds to FAP. In some embodiments, the two or more antibodies comprise an antibody that binds to EGFR and an antibody that binds to FAP. In some embodiments, the VACV is selected from GLV-1h444 and GLV-1h446. In some embodiments, the method further comprises administering an additional cancer therapy. In some embodiments, the additional cancer therapy is selected from: radiation therapy, chemotherapy, immunotherapy, phototherapy, and a combination thereof. In some embodiments, the tumor is selected from: glioblastoma, breast carcinoma, lung carcinoma, prostate carcinoma, colon carcinoma, ovarian carcinoma, neuroblastoma, central nervous system tumor, and melanoma. In some embodiments, the virus is intravenously delivered to the subject, In some embodiments, the virus is intravenously delivered directly into a tumor, or delivered to the subject within the region of a tumor. In some embodiments, the method further comprises providing to the subject at least one additional virus that can infect a cell in the tumor, wherein the virus expresses one or more: (i) antibodies targeting the tumor microenvironment (TME) or (ii) stimulatory or inhibitory proteins targeting the TME, wherein the at least one additional virus expresses one or more (i) antibodies targeting the TME or (ii) stimulatory or inhibitory proteins targeting the TME different from those expressed by the virus expressing (i) antibodies targeting the tumor microenvironment (TME) or (ii) stimulatory or inhibitory proteins targeting the TME.

Disclosed herein, in some embodiments, is a recombinant virus that can infect a cell in a solid tumor, wherein the virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein that targets the TME. In some embodiments, the virus is an oncolytic virus. In some embodiments, the oncolytic virus is a vaccinia virus. In some embodiments, the virus is a replication-competent oncolytic vaccinia virus (VACV). In some embodiments, the virus expresses two or more antibodies. In some embodiments, the two or more antibodies are selected from the group consisting of: (i) an antibody that binds to a protein that stimulates angiogenesis and/or vascularization, (ii) an antibody that binds to a receptor tyrosine kinase selected from the group consisting of epidermal growth factor receptor (EGFR), Her2/c-neu, Her3 and Her4, and (iii) an antibody that binds to a protein involved in the development of epithelial-mesenchymal interactions. In some embodiments, the virus comprises two or more heterologous nucleic acids encoding or expressing two or more antibodies selected from the group consisting of: an antibody that binds to vascular endothelial growth factor (VEGF), an antibody that binds to epidermal growth factor receptor (EGFR), and an antibody that binds to fibroblast activation protein (FAP). In some embodiments, one of the two or more antibodies is an antibody that binds to VEGF. In some embodiments, the antibody that binds to VEGF is G6-31. In some embodiments, one of the two or more antibodies is an antibody that binds to EGFR. In some embodiments, the antibody that binds to EGFR is anti-EGFRVHH. In some embodiments, one of the two or more antibodies is an antibody that binds to FAP. In some embodiments, the antibody that binds to FAP is M036. In some embodiments, the two or more antibodies comprise an antibody that binds to VEGF and an antibody that binds to EGFR. In some embodiments, the two or more antibodies comprise an antibody that binds to VEGF and an antibody that binds to FAP. In some embodiments, the two or more antibodies comprise an antibody that binds to EGFR and an antibody that binds to FAP. In some embodiments, the VACV is selected from GLV-1h444 and GLV-1h446. In some embodiments, the virus is a lister strain. In some embodiments, the A34R gene is replaced by the A34R gene from another vaccinia virus strain. In some embodiments, the A34R gene is replaced by the A34R gene from vaccinia IHD-J strain. In some embodiments, the virus comprises deletion of the A35R gene. In some embodiments, the virus further comprises an additional heterologous nucleic acid molecule encoding a diagnostic or therapeutic protein. In some embodiments, the additional heterologous nucleic acid molecule encodes a diagnostic protein. In some embodiments, the diagnostic protein is selected from among a luciferase, a fluorescent protein, an iron storage molecule, an iron transporter, an iron receptor or a protein that binds a contrasting agent, chromophore or a compound or detectable ligand that can be detected. In some embodiments, the additional heterologous nucleic acid molecule encodes a therapeutic protein. In some embodiments, the therapeutic protein is selected from among a cytokine, a chemokine, an immunomodulatory molecule, an antigen, a single chain antibody, antisense RNA, prodrug converting enzyme, siRNA, angiogenesis inhibitor, a toxin, an antitumor oligopeptides, a mitosis inhibitor protein, an antimitotic oligopeptide, an anti-cancer polypeptide antibiotic, and tissue factor.

Disclosed herein, in some embodiments, is a host cell comprising a recombinant virus as disclosed herein.

Disclosed herein, in some embodiments, is a tumor cell comprising a recombinant virus as disclosed herein.

Disclosed herein, in some embodiments, is a mammalian organism comprising or infected by the recombinant virus as disclosed herein.

Further disclosed herein is the use of a recombinant vaccinia virus, as disclosed herein, for the treatment of a tumor in a subject.

Disclosed herein, is the use of a vaccinia virus, as disclosed herein, for preparation of a pharmaceutical composition for the treatment of a tumor in a subject. In some embodiments, the pharmaceutical composition further comprises an anti-cancer compound.

Also disclosed herein is the use of a recombinant virus that can infect a cell in a tumor, or a cell comprising a virus that can infect a cell in a tumor, in a method for treating a solid tumor in a subject, comprising administering to the subject the recombinant virus that can infect a cell in a tumor, or the cell comprising a virus that can infect a cell in a tumor, wherein the virus expresses: (i) two or more antibodies that target the tumor microenvironment (TME); (ii) two or more of a stimulatory or inhibitory protein targeting the TME, and (iii) at least one antibody that targets the TME and at least one of a stimulatory or inhibitory protein targeting the TME.

### BRIEF DESCRIPTION OF THE DRAWINGS

FIG. 1 exemplifies a schematic representation of antibodies and the new VACVs. (a) Schematic diagrams of anti-EGFRVHHFLAG, anti-VEGF scAb (GLAF-2), and anti-FAP scAb (GLAF-5) constructs. (b) Genomic structures of the new recombinant VACVs along with their parental virus. GLV-1h442 and GLV-1h282 were derived from GLV-1h68 by replacing the lacZ expression cassette at the J2R locus with the anti-EGFRVHHFLAG and GLAF-5 cassettes, respectively, each under the control of the PSEL promoter. GLV-1h164 was derived from GLV-1h68 by replacing the lacZ expression cassette at the J2R locus with the hNET under the PSE promoter and the gusA expression cassette at A56R locus with the GLAF-2 cassette under the PSL promoter. GLV-1h444 and GLV-1h446 were derived from GLV-1h164 by replacing the hNET expression cassette at the J2R locus with the anti-EGFRVHHFLAG and the GLAF-5 expression cassette, respectively, each under the control of the VACV PSEL promoter. All viruses contain the ruc-gfp expression cassette at the F14.5L locus. PSE, PSEL, PSL, P11, and P7.5 are VACV synthetic early, synthetic early/late, synthetic late, 11K, and 7.5K promoters, respectively.

FIG. 2 exemplifies virally expressed individual therapeutic antibodies targeting the TME significantly enhance virotherapy in A549 and DU145 tumor xenograft models. (a) An antibody targeting VEGF expressed from GLV-1h164 significantly enhanced virotherapy. Mice bearing A549 xenograft tumors (n≧7) were treated with virus alone, Avastin alone, PBS alone, or virus in combination with Avastin. A single dose of virus (2×10⁶ pfu/mouse) was given intravenously (i.v.) when tumor volumes reached 450 mm³. Avastin was administered i.p. at a dose of 5 mg/kg, twice per week for 5 weeks, starting at 10 dpi. The arrows indicate the beginning and end of Avastin treatment. Statistical analysis was performed using one-way ANOVA (***P<0.001, **P<0.01, *P<0.05). Stars indicate the comparison of the GLV-1h68 group with the GLV-1h164 group (black) and with the GLV-1h68+Avastin group (open). αV indicates anti-VEGF. (b) Antibody targeting EGFR expressed from GLV-1h442 significantly enhanced virotherapy. Mice bearing A549 xenograft tumors (n≧7) were treated with virus alone, Erbitux alone, PBS alone, or virus in combination with Erbitux. A single dose of virus (2×10⁶ pfu/mouse) was given i.v. when tumor volumes reached 450 mm³. Erbitux was administered i.p. at a dose of 3 mg/kg, twice per week for 5 weeks, starting at 10 dpi. The arrows indicate the beginning and end of Erbitux treatment. Statistical analysis was performed using one-way ANOVA (**P<0.01, *P<0.05). Stars indicate the comparison of the GLV-1h68 group with the GLV-1h442 group. αE indicates anti-EGFR. (c) Antibody targeting FAP expressed from GLV-1h282 significantly enhanced virotherapy. Mice bearing A549 xenograft tumors (n≧7) were injected i.v. with a single dose of GLV-1h68 or GLV-1h282 (2×10⁶ pfu/mouse) when tumor volumes reached 450 mm3. Statistical analysis was performed using one-way ANOVA (**P<0.01, *P<0.05). Stars indicate the comparison of the GLV-1h68 group with the GLV-1h282 group. αF indicates anti-FAP. (d) DU145 tumor-bearing mice (n=5) were injected i.v. with each VACV strain (2×10⁶ pfu/mouse) when tumor volumes reached 450 mm3, and tumor volumes were monitored weekly thereafter. Statistical analysis was performed using one-way ANOVA (***P<0.001, **P<0.01, *P<0.05). Stars indicate the comparison of the GLV-1h68 group with the GLV-1h164 group (black), GLV-1h282 group (open), and GLV-1h442 group (gray).

FIG. 3 exemplifies Influences of intratumorally expressed antibodies targeting VEGF, EGFP, and FAP on the TME. (a) Effect of virus treatment on tumor vasculature in DU145 tumors (n=3). Sections were stained for CD31 expression (red). GFP expression (green) indicates virus infection. (b) Quantitative analysis of blood vessels was performed by counting CD31+ blood vessels in eight non-overlapping microscopic fields per slide. Statistical analysis was performed with a two-tailed unpaired Student's t-test (**P<0.01, *P<0.05). (c) Effect of virus treatment on cell proliferation in DU145 tumors (n=3). Sections were stained for Ki67 expression (red). GFP expression (green) indicates virus infection. Scale bars represent 1 mm in a, c. (d) Quantitative analysis of cell proliferation was performed by counting Ki67+ cells in eight non-overlapping microscopic fields per slide. Statistical analysis was performed with a two-tailed unpaired Student's t-test (**P<0.01, *P<0.05). (e) Immunohistochemical characterization of viral replication and stromagenesis. Formalin-fixed and paraffin-embedded tumor tissues (n=4-5 per group) were cut into 5-μm sections and H&E staining was performed. Adjacent sections were stained with anti-A27 for VACV, anti-CD31 for blood vessels and anti-FAP for FAP+ stromal cells. (f) Bar graphs show mean numbers of CD31+ cells and FAP+ stromal clusters in infected or uninfected areas. CD31+ and FAP+ stromal clusters in five non-overlapping microscopic fields (100× magnification) per tumor were counted. Statistical analysis was performed with a two-tailed unpaired Student's t-test (**P<0.01, *P<0.05).

FIG. 4 exemplifies how FaDu tumor growth was significantly inhibited by GLV-1h282 expressing anti-FAP scab. However, FaDu tumors did not respond to treatment with GLV-1h68.

FIG. 5 exemplifies how each recombinant VACV expressed the intended antibodies.

FIG. 6 exemplifies how expression of two antibodies did not show negative effects on viral replication efficiency. Viral replication assays were performed in A549 cells at a multiplicity of infection (MOI) of 0.01.

FIG. 7 exemplifies virally expressed two therapeutic antibodies targeting the TME further improve virotherapy. (a) Enhanced therapeutic effects of GLV-1h444 in A549 tumor-bearing nude mice. Mice (n≧7) were treated with virus alone, Avastin+Erbitux, PBS alone, or virus in combination with Avastin and Erbitux. A single dose of virus (2×106 pfu/mouse) was given i.v. when tumor volumes reached 450 mm3. Avastin and Erbitux were administered i.p. at doses of 5 mg/kg and 3 mg/kg, respectively, twice per week for 5 weeks, starting at 10 dpi. The arrows indicate the beginning and end of Avastin and Erbitux treatment. Statistical analysis was performed using one-way ANOVA (***P<0.001, **P<0.01, *P<0.05). Stars indicate the comparison of the GLV-1h68 group with the GLV-1h444 group (black), the GLV-1h68+Avastin+Erbitux group (open), and the PBS+Avastin+Erbitux group (gray). (b) Enhanced therapeutic effects of GLV-1h446 compared with its parental viruses in A549 tumor-bearing nude mice. Mice (n≧7) were i.v. injected with each VACV strain alone at a dose of 2×106 pfu/mouse when tumor volumes reached 450 mm3. Statistical analysis was performed using one-way ANOVA (***P<0.001, **P<0.01, *P<0.05). Stars indicate the comparison of the GLV-1h68 group with the GLV-1h446 group (black). (c) Enhanced therapeutic effects of GLV-1h444 and GLV-1h446 compared with their parental viruses in DU145 tumor-bearing mice. Mice (n=5) were injected i.v. with each VACV strain alone at a dose of 2×10⁶ pfu/mouse when tumor volumes reached 450 mm3. Statistical analysis was performed using one-way ANOVA (***P<0.001, **P<0.01, *P<0.05). Stars indicate the comparison of the GLV-1h68 group with the GLV-1h444 group (black) and with the GLV-1h446 group (open). (d-f) Detection of antibodies in mice sera and the change of tumor volumes in mice bearing A549 tumors after VACV treatment. Blood samples were collected retro-orbitally at 7, 21, and 35 dpi. The concentration of antibodies in mice sera was determined by ELISA with precoated FAP, EGFR, and VEGF plates. Tumors were measured with a digital caliper. Statistical analysis was performed with a two-tailed unpaired Student's t-test (***P<0.001).

FIG. 8 exemplifies tumor growth curves of individual mice bearing A549 tumors.

FIG. 9 exemplifies viral biodistribution in different organs and tumors in A549 tumor-bearing mice at 14 dpi as determined by standard viral plaque assays.

FIG. 10 exemplifies an assessment of possible adverse effect of antibody-expressing VACV administration in mice. The assessment was made by evaluating the change in net body weight over the course of treatment. In both A549 and DU145 tumor xenograft models, no significant change in the mean net body weight was observed for any of the treated or control groups

FIG. 11 exemplifies virally expressed two antibodies by GLV-1h444 and GLV-1h446 contribute to the suppression of cell proliferation in tumors. (a) Suppression of cell proliferation by VACVs in DU145 tumors (n=3). GFP expression (green) indicates virus infection. Cell proliferation was examined by staining with anti-Ki67 antibody (red). (b, c) Quantitative analysis of cell proliferation in uninfected or infected areas was performed by counting Ki67+ cells in eight non-overlapping microscopic fields per slide. Statistical analysis was performed using a two-tailed unpaired Student's t-test (**P<0.01, *P<0.05).

FIG. 12 exemplifies virally expressed two antibodies by GLV-1h444 and GLV-1h446 contribute to the suppression of angiogenesis in tumors. (a) Reduced tumor vasculature by VACVs in DU145 tumors (n=3). Sections were stained with anti-CD31 antibody (red). GFP expression (green) indicates virus infection. (b, c) Quantitative analysis of blood vessels in uninfected or infected areas was performed by counting CD31+ cells in eight non-overlapping microscopic fields per slide. Statistical analysis was performed using a two-tailed unpaired Student's t-test (***P<0.001, **P<0.01, *P<0.05). Scale bars indicate 1 mm in a, d.

### DETAILED DESCRIPTION

In spite of great advances in anticancer treatments over the past 30 years, cancer remains the leading cause of death around the world. Overlooking the important role of tumor microenvironment (TME) in cancer growth and metastasis may be one of the reasons that fully-effective cancer treatment remains elusive. Angiogenesis and hyper-proliferation of cells in the stroma of tumors not only support the growth of cancer but also contribute to its development, for example, in metastasis.

Factors within the TME such as vascular endothelial growth factor (VEGF), epidermal growth factor receptor (EGFR), and fibroblast activation protein (FAP) play crucial roles in cancer initiation and development. The high-level expression of VEGF or EGFR correlates with poor prognosis in patients with breast, colon, lung, head and neck, and other cancers. The anti-VEGF monoclonal antibody (mAb), bevacizumab (Avastin), was approved by the US Food and Drug Administration (FDA) in 2004 for the treatment of metastatic colon cancer and subsequently other metastatic cancers. In the same year, anti-EGFR mAb, cetuximab (Erbitux), was also approved by the FDA for the treatment of metastatic colon cancer. However, the clinical efficacy of Avastin and Erbitux has been somewhat limited possibly due to poor tumor penetration and rapid clearance of the mAbs from circulation, requiring the administration of high doses at frequent intervals and extensive durations, also making the therapies extremely costly.

Improvements in the pharmacodynamic properties of current mAb therapeutics and identification of additional functionalities targeting the TME could be greatly beneficial. For example, G6-31 is an improved anti-VEGF antibody derived from a phage display library with better binding affinity and enhanced therapeutic efficacy in animal models than Avastin. To improve tumor penetration, a single-domain antibody of 15 kDa against EGFR from a llama has been recently developed (termed anti-EGFRVHH). This llama nanobody was nonimmunogenic in mice and was proven to block binding of EGF to EGFR, thereby inhibiting EGFR signaling and showing the specific tumor targeting. Anti-EGFRVHH is used for molecular imaging and therapeutic applications. FAP (also known as seprase), a highly conserved protein, is richly expressed particularly in the stroma of aggressive cancers. A high-level of FAP expression is correlated with cancer progression. M036, a species-cross-reactive FAP-specific single-chain antibody (scAb), was isolated by sequential phage display and was shown to bind FAP on stromal cells of different human carcinomas and the murine host stroma in human tumor xenografts. The therapeutic potential of M036 has not yet been evaluated.

The replication competent oncolytic vaccinia virus (VACV) GLV-1h68 locates, replicates, and lyses tumor cells in human xenograft nude mouse models after administration of a single dose. Additionally, recombinant VACVs can be genetically modified to express functional transgenes, including scAbs. It has been previously shown that VACVs expressing the anti-VEGF scAb GLAF-1, designed according to the sequence of G6-31, significantly improved anti-cancer therapeutic efficacy in mice compared with the parental virus, GLV-1h68. The therapeutic efficacy was further enhanced in combination with radiation therapy.

Thus, new recombinant VACVs expressing novel TME-targeted antiproliferative activities were constructed by encoding a scAb against FAP (GLV-1h282) and a single-domain antibody against EGFR (GLV-1h442). VACVs expressing these individual antibodies significantly suppressed tumor growth in xenograft tumor models, verifying the functionality and therapeutic activity of the virally expressed antibodies. Lastly, additional recombinant VACVs were created encoding two antibodies with both antiproliferative and antiangiogenic activities targeting VEGF and EGFR (GLV-1h444) or VEGF and FAP (GLV-1h446). The new VACVs expressing the TME-targeted antibodies, either singly or in combination significantly enhanced the antitumor efficacy of oncolytic virotherapy.

Moreover, treatment of tumors in mice with the two antibody-expressing VACVs, GLV-1h444 (anti-EGFR and anti-VEGF) or GLV-1h446 (anti-FAP and anti-VEGF), was surprisingly superior to the concomitant treatment with GLV-1h68 in combination with continuous administration of Avastin (anti-VEGF) and Erbitux (anti-EGFR). The antiproliferative and antiangiogenic effects of the virally expressed antibodies were also apparent in tumors beyond the areas directly infected by the virus, demonstrating that the scAbs are capable of tumor permeation, while locally expressed. Thus, our results demonstrated that oncolytic virotherapy with VACV was significantly enhanced by coexpression of virus-encoded antibodies with antiproliferative and antiangiogenic activities targeting the TME. Additionally, the enhanced treatment effects were achieved by a single administration of the replication-competent, recombinant VACVs.

Thus, new recombinant VACVs expressing antibodies targeting VEGF, EGFR, and FAP, either alone or in combination, significantly enhanced oncolytic virotherapy in preclinical animal models. The therapeutic efficacy of GLV-1h164, GLV-1h442, and GLV-1h282, each expressing a scAb, was significantly better than that of their parental virus GLV-1h68, indicating that oncolytic virotherapy can be improved by viral expression of individual antibodies against VEGF to reduce angiogenesis, EGFR to suppress cell proliferation, or FAP to reduce angiogenesis and suppress recruitment of MSCs. Moreover, the therapeutic efficacy was further enhanced by expressing two antibodies in one VACV strain. The therapeutic efficacy of GLV-1h444 that expresses antibodies targeting both VEGF and EGFR was significantly better than that of the combination treatment with Avastin and Erbitux and was also superior to treatment with GLV-1h68 in combination with Avastin and Erbitux.

High-level expression of VEGF, EGFR, and FAP in tumors is associated with poor prognosis. Despite promising results in preclinical trials, Avastin and Erbitux have shown only limited clinical efficacy, partially owing to the poor penetration and low tumor targeting of the antibodies as well as rapid clearance from the circulation after systemic administration. The oncolytic VACV not only specifically targets and destroys tumor cells but also mediates local production of therapeutic proteins in colonized tumors, thus circumventing the limitations associated with the use of antibody therapeutics. The continuous presence of antibodies was demonstrated in the sera of mice treated with VACVs expressing either one or two antibodies. The antibodies were detected in higher amounts in the early phase (7 and 21 dpi) than in the later phase (35 dpi) following injection of the virus when tumors had already started to shrink. In addition, anti-FAP (GLAF-5) and anti-EGFRVHHFLAG antibodies occurred in higher amounts in the sera of mice treated with GLV-1h282 and GLV-1h442, respectively, than was anti-VEGF (GLAF-2) antibody in the sera of mice treated with GLV-1h164. This was consistent with the higher viral titers of GLV-1h282 and GLV-1h442 than GLV-1h164 in tumors. Although the viral titer of GLV-1h68 in tumors was significantly higher than that of GLV-1h164 (P=0.02), GLV-1h164 replicated slightly faster than GLV-1h68 in culture, suggesting that the expression of GLAF-2 decreased viral replication in tumors. The expression of the antibodies targeting EGFR or FAP had no negative effect on viral replication in tumors.

It is well known that cancer progression is due to uncontrolled growth of cancer cells. Treatment with GLV-1h68 suppressed cell proliferation in tumors, evident in the dramatic decrease in the number of Ki67+ cells in the infected areas, consistent with the intrinsic property of VACV infection. Notably, treatment with GLV-1h442 and GLV-1h444, expressing anti-EGFR nanobody, significantly reduced the number of Ki67+ cells not only in the infected areas but also in the uninfected areas, suggesting that the anti-EGFR nanobody secreted from the infected cells spread into uninfected areas, suppressing the proliferation of EGFR+ cells. Although treatment with GLV-1h164, expressing anti-VEGF antibody, or GLV-1h282, expressing anti-FAP antibody, significantly suppressed proliferation in the infected areas of the tumor, the effect was only slight, but not significant, in the uninfected areas. Importantly, treatment with GLV-1h446, expressing both anti-VEGF and anti-FAP, significantly suppressed proliferation in both the infected and uninfected areas, demonstrating the added inhibitory effect of two antibody expressions on cell growth.

Cancer growth and development also requires a continuous supply of nutrients through blood vessels. A reduction of blood vessel density (BVD) was observed in the infected areas, but not in the uninfected areas of GLV-1h68-colonized tumors, suggesting that VACV infection itself can lead to the destruction of tumor vasculature. The expression of anti-VEGF scAb not only enhanced blood vessel destruction in the infected areas but also caused a significant decrease in BVD in the uninfected areas. Thus, like anti-EGFR nanobody, anti-VEGF scAb can also spread from infected areas to uninfected areas. Therapies targeting FAP inhibit tumor growth partially through suppressing tumor angiogenesis. Virally expressed anti-FAP scAb significantly decreased BVD in both the infected and uninfected areas in the FaDu tumor xenograft model, and in the infected areas in the DU145 tumor xenograft model. Although the expression of the anti-EGFR nanobody did not significantly affect BVD in either infected or uninfected areas in the DU145 tumor xenograft model, it has been reported that EGFR promotes tumor angiogenesis.

FAP is one of the markers expressed by cancer-associated fibroblasts (CAFs) that support tumor growth. The ablation of FAP+ CAFs has been shown to suppress tumor growth. Treatment of mice bearing FaDu tumor xenografts with GLV-1h282, expressing anti-FAP scAb, resulted in a great reduction in the number of FAP+ cells in both the infected and uninfected areas of the tumor compared with treatment with GLV-1h68. The enhanced antitumor effects of GLV-1h282 are likely attributed to an effect on CAFs, rather than on the cancer cells, since FaDu tumor cells do not express FAP.

Thus, oncolytic VACV infection itself greatly reduced cell proliferation and vascularity of colonized tumors. These antitumor effects were enhanced significantly by the expression of scAbs against VEGF, EGFR, and FAP in recombinant VACVs either alone or in combination. The effects of coexpression of the antibody or antibodies were either comparable or superior to the treatment with VACV combined with the clinical antibodies, with the benefit of single administration and localized intratumoral delivery. The therapeutic effect combined viral oncolysis of the infected tumor cells with antitumor alterations of the TME. To our knowledge, this is the first report demonstrating that virally expressed antibodies against EGFR and FAP singly enhanced oncolytic virotherapy and, in combination with anti-VEGF antibody, further improved antitumor therapeutic efficacy.

### Definitions

As used herein, a subject includes any animal for whom diagnosis, screening, monitoring or treatment is contemplated. Animals include mammals such as primates and domesticated animals. In some embodiments, the subject is a mammal. In some embodiments, the subject is a mammal selected from a mouse, a rat, a rabbit, a dog or a cat. In some embodiments, the subject is a primate. An exemplary primate is human. A patient refers to a subject such as a mammal, primate, human, or livestock subject afflicted with a disease condition or for which a disease condition is to be determined or risk of a disease condition is to be determined.

As used here, the term “antibody” is used in the broadest sense and specifically covers monoclonal antibodies (including full length monoclonal antibodies), polyclonal antibodies, multispecific antibodies (e.g., bispecific antibodies), bi-specific T cell engagers (BiTE) antibodies, and antibody fragments (e.g., single-chain, nanobodies, etc.) so long as they exhibit the desired biological activity. In some embodiments, the antibody is a polyclonal antibody. In some embodiments, the antibody is a monoclonal antibody. In some embodiments, the antibody is a Fab fragment. In some embodiments, the antibody is a single-chain antibody. In some embodiments, the antibody is a nanobody. In some embodiments, the antibody is selected from a Fab, Fv, F(ab′)2, a scFV, a diabody and a bispecific antibody. In some embodiments, the antibody is a humanized antibody. In some embodiments, any of the antibody embodiments recited herein are specifically excluded.

As used herein, “virus” refers to any of a large group of entities referred to as viruses. Viruses typically contain a protein coat surrounding an RNA or DNA core of genetic material, but no semipermeable membrane, and are capable of growth and multiplication only in living cells. Viruses for use in the methods provided herein include, but are not limited, to a poxvirus, adenovirus, herpes simplex virus, Newcastle disease virus, vesicular stomatitis virus, mumps virus, influenza virus, measles virus, reovirus, human immunodeficiency virus (HIV), hanta virus, myxoma virus, cytomegalovirus (CMV), lentivirus, and any plant or insect virus. In some embodiments, any of the virus embodiments recited herein are specifically excluded.

As used herein, the term “viral vector” is used according to its art-recognized meaning. It refers to a nucleic acid vector construct that includes at least one element of viral origin and can be packaged into a viral vector particle. The viral vector particles can be used for the purpose of transferring DNA, RNA or other nucleic acids into cells either in vitro or in vivo. Viral vectors include, but are not limited to, retroviral vectors, vaccinia vectors, lentiviral vectors, herpes virus vectors (e.g., HSV), baculoviral vectors, cytomegalovirus (CMV) vectors, papillomavirus vectors, simian virus (SV40) vectors, semliki forest virus vectors, phage vectors, adenoviral vectors, and adeno-associated viral (AAV) vectors.

As used herein, “hematologic malignancy” refers to tumors of the blood and lymphatic system (e.g. Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma).

### Recombinant Viruses and Methods of Use

Disclosed herein, in some embodiments, is a method for treating a solid tumor, comprising providing to a subject a recombinant virus that can infect a cell in the tumor, or a cell comprising a virus that can infect a cell in the tumor, wherein the virus expresses two or more: (i) antibodies targeting the tumor microenvironment (TME) or (ii) stimulatory or inhibitory proteins targeting the TME. In some embodiments, the virus is an oncolytic virus. In some embodiments, the oncolytic virus is a vaccinia virus. In some embodiments, the virus is a replication-competent oncolytic vaccinia virus (VACV). In some embodiments, the method further comprising providing to the subject an additional virus, wherein the additional virus expresses one or more: (i) antibodies targeting the tumor microenvironment (TME) or (ii) stimulatory or inhibitory proteins targeting the TME, wherein the at least one additional virus expresses one or more (i) antibodies targeting the TME or (ii) stimulatory or inhibitory proteins targeting the TME different from those expressed by the virus expressing (i) antibodies targeting the tumor microenvironment (TME) or (ii) stimulatory or inhibitory proteins targeting the TME

Thus, provided herein are viruses for therapeutic and diagnostic use, including recombinant vaccinia viruses that contain a heterologous nucleic acid molecule that encodes at least two therapeutic gene products (i.e., two or more: (i) antibodies targeting the tumor microenvironment or (ii) stimulatory or inhibitory proteins targeting the tumor microenvironment). Such therapeutic gene products can be operably linked to any suitable promoter (e.g., a vaccinia promoter, such as a vaccinia early promoter, a vaccinia intermediate promoter, a vaccinia early/late promoter and a vaccinia late promoter).

In some embodiments, the two or more antibodies targeting the tumor microenvironment (TME) are selected from the group consisting of: (i) an antibody that binds to a protein that stimulates angiogenesis and/or vascularization, (ii) an antibody that binds to a receptor tyrosine kinase selected from the group consisting of epidermal growth factor receptor (EGFR), Her2/c-neu, Her3 and Her4 (the Erb family), and (iii) an antibody that binds to a protein involved in the development of epithelial-mesenchymal interactions. Exemplary proteins that stimulate angiogenesis and/or vascularization include paracrine factors (including angiogenin, vascular endothelial growth factor (VEGF), fibroblast growth factor (FGF), and transforming growth factor-β (TGF-β) and integrins. Proteins involved in the development of the epithelial mesenchymal transition include fibroblast activation protein (FAP), HSP47, collagen 1, collagen 2, vimentin FSP1, DDR2, N-Cadherin, Snail, Slug, and Twis, OB-cadherin, integrins, Syndecan-1, FSP-1, beta-catenin, fibronectin, laminin 5, ZEB1, LEF-1, Ets-1, FOXC2, and Goosecoid. In some embodiments, the two or more antibodies include an antibody specific for VEGF, angiogenin, FGF, TGF-β, or an integrin (e.g., αᵥβ3 , αᵥβ5 or α₅β1). In some embodiments, the two or more antibodies include an antibody specific for EGFR, Her2/c-neu, Her3 or Her4. In some embodiments, the two or more antibodies include an antibody specific for FAP, HSP47, collagen 1, collagen 2, vimentin FSP1, DDR2, N-Cadherin, Snail, Slug, and Twis, OB-cadherin, integrins, Syndecan-1, FSP-1, beta-catenin, fibronectin, laminin 5, ZEB1, LEF-1, Ets-1, FOXC2, or Goosecoid. In some embodiments, any of the antigen embodiments listed in this paragraph are specifically excluded.

In some embodiments, the two or more antibodies are selected from the group consisting of: an antibody that binds to vascular endothelial growth factor (VEGF), an antibody that binds to epidermal growth factor receptor (EGFR), and an antibody that binds to fibroblast activation protein (FAP). In some embodiments, one of the two or more antibodies is an antibody that binds to VEGF. In some embodiments, the antibody that binds to VEGF is G6-31. In some embodiments, the antibody that binds to VEGF is selected from: 4G3, ab52917, ab68334, ab46154, A-20, OTI4E3, Ab-3. SP28. bevacizumab, ranibuzumab, pazopanib, sorafenib, sunitinib, vandeteanib, cabozantinib, ponatinib, axitinib, and aflibercept, and antibodies capable of binding to the same epitope as any of these antibodies. In some embodiments, one of the two or more antibodies is an antibody that binds to EGFR. In some embodiments, the antibody that binds to EGFR is anti-EGFRVHH. In some embodiments, the antibody that binds to EGFR is selected from cetuximab, matuzumab, panitumumab, necitumumab, nimotuzumab, trastuzumab, zalutumumab, 528, SC-03, DR8.3, DH8.3, L8A4, Y10, ICR62, ABX-EGF, EMD72000, MM-151, Sym004, mAB 806, and antibodies capable of binding to the same epitope as any of these antibodies. In some embodiments, one of the two or more antibodies is an antibody that binds to FAP. In some embodiments, the antibody that binds to FAP is M036. In some embodiments, the antibody that binds to FAP is ab54651, vF19, ESC11, ESC14, F11-24, SS-13, D394, MAb clone 427819, MO5, M02, M01, LS-C348807, 2F2. and antibodies capable of binding to the same epitope as any of these antibodies. In some embodiments, the two or more antibodies comprise an antibody that binds to VEGF and an antibody that binds to EGFR. In some embodiments, the two or more antibodies comprise an antibody that binds to VEGF and an antibody that binds to FAP. In some embodiments, the two or more antibodies comprise an antibody that binds to EGFR and an antibody that binds to FAP. In some embodiments, any of the specific antibodies listed in this paragraph, or antibodies binding to the same epitope as the specific antibodies listed int his paragraph, as specifically excluded.

In some embodiments, the invention disclosed herein does not encompass a virus that expresses an antibody against VEGF, but does not express an antibody or protein that binds to a receptor tyrosine kinase selected from the group consisting of epidermal growth factor receptor (EGFR), Her2/c-neu, Her3 and Her4 (the Erb family), and does not express an antibody or protein that binds to a protein involved in the development of epithelial-mesenchymal interactions.

Exemplary oncolytic viruses include vaccinia virus, vesicular stomatitis virus (VSV), Newcastle disease virus (NDV), retrovirus, reovirus, measles virus, Sinbis virus, influenza virus, herpes simplex virus (HSV), vaccinia virus, and adenovirus.

In some embodiments, a virus disclosed herein is attenuated. Techniques for attenuating viruses are known in the art.

Exemplary viruses provided herein include recombinant vaccinia viruses that contain a modified hemagglutinin (HA) gene, thymidine kinase (TK) gene, and F14.5L gene, where one or more of the modifications comprises insertion of a heterologous non-coding nucleic acid molecule into the HA gene locus, TK gene locus, or F14.5L gene locus. In such viruses, a functional HA, TK, and F14.5L polypeptide is not expressed.

Exemplary viruses provided herein for therapeutic and diagnostic use also include Western Reserve (WR), Copenhagen, Tashkent, Tian Tan, Lister, Wyeth, IHD-J, and IHD-W, Brighton, Ankara, MVA, Dairen I, LIPV, LC16M8, LC16MO, LIVP, WR 65-16, Connaught, New York City Board of Health. LIVP vaccinia viruses described herein for use in the methods described herein include GLV-1h22, GLV-1h68, GLV-1i69, GLV-1h70, GLV-1h71, GLV-1h72, GLV-1h73, GLV-1h75, GLV-1h81, GLV-1h82, GLV-1h83, GLV-1h84, GLV-1h85, GLV-1h86, GLV-1j87, GLV-1j88, GLV-1j89, GLV-1h90, GLV-1h91, GLV-1h92, GLV-1h96, GLV-1h97, GLV-1h98, GLV-1h104, GLV-1h105, GLV-1h106. Exemplary LIVP vaccinia viruses provided herein for use in the methods described herein include GLV-1h107, GLV-1h108 and GLV-1h109. In some embodiments, the virus for use in the methods described is selected from GLV-1h107, GLV-1h108 and GLV-1h109.

In some embodiments, a virus, as claimed, is selected from GLV-1h444 and GLV-1h446.

In some embodiments, viruses provided herein for therapeutic and diagnostic use include recombinant vaccinia viruses that contain a heterologous nucleic acid molecule that encodes a detectable protein or a protein capable of inducing a detectable signal. Exemplary of such proteins are luciferases, such as a click beetle luciferase, a Renilla luciferase, or a firefly luciferase, fluorescent proteins, such as a GFP or RFP, or proteins that can bind a contrasting agent, chromophore, or a compound or ligand that can be detected, such as a transferrin receptor or a ferritin. Provided herein are recombinant Lister strain vaccinia viruses as claimed that express click beetle luciferase (CBG99) and RFP (e.g., GLV-1h84).

Described herein are viruses for therapeutic and diagnostic use that contain a heterologous nucleic acid molecule that encodes two or more diagnostic or therapeutic gene products, where the gene products are linked by a picornavirus 2A element. In one example described herein, the recombinant vaccinia virus contains a heterologous nucleic acid molecule that encodes CBG99 is linked by a picornavirus 2A element to a second heterologous nucleic acid molecule that encodes RFP (e.g., GLV-1h84).

In some embodiments, described herein are recombinant vaccinia viruses for therapeutic and diagnostic use that contain a replacement of the A34R gene with the A34R gene from another vaccinia virus strain. Provided herein is a Lister strain vaccinia virus as claimed, where the A34R gene is replaced by the A34R gene from vaccinia IHD-J strain (e.g., GLV-1 i69). Such replacement increases the extracellular enveloped virus (EEV) form of vaccinia virus and increases the resistance of the virus to neutralizing antibodies.

Described herein are recombinant vaccinia viruses for therapeutic and diagnostic use that contain deletion of the A35R gene.

Described herein are recombinant vaccinia viruses for therapeutic and diagnostic use that can be further modified by addition of one or more additional heterologous nucleic acid molecules that encode a therapeutic protein, a detectable protein or a protein capable of inducing a detectable signal. Exemplary of such proteins are luciferases, such as a click beetle luciferase, a Renilla luciferase, or a firefly luciferase, fluorescent proteins, such as a GFP or RFP, or proteins that can bind a contrasting agent, chromophore, or a compound or ligand that can be detected, such as a transferrin receptor or a ferritin. In some embodiments, the diagnostic protein is selected from among a luciferase, a fluorescent protein, an iron storage molecule, an iron transporter, an iron receptor or a protein that binds a contrasting agent, chromophore or a compound or detectable ligand that can be detected.

Also included in such methods are insertion of heterologous nucleic acid molecules that encode a therapeutic gene product, such as a cytokine, a chemokine, an immunomodulatory molecule, a single chain antibody, antisense RNA, siRNA, prodrug converting enzyme, a biological toxin, an antitumor oligopeptide, an anti-cancer polypeptide antibiotic, angiogenesis inhibitor, or tissue factor. Exemplary antigens include tumor specific antigens, tumor-associated antigens, tissue-specific antigens, bacterial antigens, viral antigens, yeast antigens, fungal antigens, protozoan antigens, parasite antigens, and mitogens. The one or more additional heterologous nucleic acid molecules that encode a therapeutic protein, a detectable protein or a protein capable of inducing a detectable signal can be operatively linked to a promoter, such as a vaccinia virus promoter. In some embodiments, therapeutic protein is selected from among a cytokine, a chemokine, an immunomodulatory molecule, an antigen, a single chain antibody, antisense RNA, prodrug converting enzyme, siRNA, angiogenesis inhibitor, a toxin, an antitumor oligopeptides, a mitosis inhibitor protein, an antimitotic oligopeptide, an anti-cancer polypeptide antibiotic, and tissue factor.

Provided herein are host cells that contain a recombinant virus as claimed. An exemplary host cell is a tumor cell that contains a recombinant virus as claimed. In some embodiments, host cells are a mammalian cell line in culture. In some embodiments, the host cells are a human cell line in culture.

Also disclosed herein is a mammalian organism comprising or infected by recombinant virus as claimed. In some embodiments, the mammalian organism is a mouse, rat, rabbit, or simian.

Provided herein are pharmaceutical compositions that contain a recombinant virus as claimed and a pharmaceutically acceptable carrier. The compositions contain an amount or concentration of the virus suitable for the intended use, such as therapy, diagnostics or both, and route of administration. Provided herein are such pharmaceutical compositions formulated for local or systemic administration. Provided herein are such pharmaceutical compositions that contain two or more viruses. Provided herein are such pharmaceutical compositions that are formulated for administration as a vaccine, such a smallpox vaccine.

Provided herein are pharmaceutical compositions for use for treating a tumor, cancer or metastasis in a subject, such as a human subject or an animal subject. Administering the pharmaceutical composition causes tumor growth to stop or be delayed, causes a reduction in tumor volume or causes the tumor to be eliminated from the subject.

The methods and pharmaceutical compositions disclosed herein can be used to treat any solid tumor or hematologic malignancy. Tumors that can be treated by the methods disclosed herein include, but are not limited to a bladder tumor, breast tumor, prostate tumor, carcinoma, basal cell carcinoma, biliary tract cancer, bladder cancer, bone cancer, brain cancer, CNS cancer, glioma tumor, cervical cancer, choriocarcinoma, colon and rectum cancer, connective tissue cancer, cancer of the digestive system, endometrial cancer, esophageal cancer, eye cancer, cancer of the head and neck, gastric cancer, intra-epithelial neoplasm, kidney cancer, larynx cancer, leukemia, liver cancer, lung cancer, lymphoma, Hodgkin's lymphoma, Non-Hodgkin's lymphoma, melanoma, myeloma, neuroblastoma, oral cavity cancer, ovarian cancer, pancreatic cancer, retinoblastoma, rhabdomyosarcoma, rectal cancer, renal cancer, cancer of the respiratory system, sarcoma, skin cancer, stomach cancer, testicular cancer, thyroid cancer, uterine cancer, and cancer of the urinary system, such as lymphosarcoma, osteosarcoma, mammary tumors, mastocytoma, brain tumor, melanoma, adenosquamous carcinoma, carcinoid lung tumor, bronchial gland tumor, bronchiolar adenocarcinoma, small cell lung cancer, non-small cell lung cancers, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumor, testicular tumor, seminoma, Sertoli cell tumor, hemangiopericytoma, histiocytoma, chloroma, granulocytic sarcoma, corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma, cystadenoma, follicular lymphoma, intestinal lymphosarcoma, fibrosarcoma, and pulmonary squamous cell carcinoma, leukemia, hemangiopericytoma, ocular neoplasia, preputial fibrosarcoma, ulcerative squamous cell carcinoma, preputial carcinoma, connective tissue neoplasia, mastocytoma, hepatocellular carcinoma, lymphoma, pulmonary adenomatosis, pulmonary sarcoma, Rous sarcoma, reticulo-endotheliosis, fibrosarcoma, nephroblastoma, B-cell lymphoma, lymphoid leukosis, retinoblastoma, hepatic neoplasia, lymphosarcoma, plasmacytoid leukemia, swimbladder sarcoma (in fish), caseous lumphadenitis, lung carcinoma, insulinoma, lymphoma, sarcoma, neuroma, pancreatic islet cell tumor, gastric MALT lymphoma and gastric adenocarcinoma. In some embodiments, the tumor is selected from: glioblastoma, breast carcinoma, lung carcinoma, prostate carcinoma, colon carcinoma, ovarian carcinoma, neuroblastoma, central nervous system tumor, and melanoma.

A pharmaceutical composition provided herein can be administered systemically, intravenously, intraarterially, intratumorally, endoscopically, intralesionally, intramuscularly, intradermally, intraperitoneally, intravesicularly, intraarticularly, intrapleurally, percutaneously, subcutaneously, orally, parenterally, intranasally, intratracheally, by inhalation, intracranially, intraprostaticaly, intravitreally, topically, ocularly, vaginally, or rectally.

The pharmaceutical composition provided herein can be administered with an anti-viral agent, such as, but not limited to, cidofovir, alkoxyalkyl esters of cidofovir, Gleevec, gancyclovir, acyclovir, and ST-26.

Provided herein are combinations as claimed that contain a pharmaceutical composition provided herein and an anticancer agent. Exemplary anticancer agents for use in combinations provided herein include, but are not limited to, a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, hyperthermia or hyperthermia therapy, a bacterium, radiation therapy or a combination thereof. Exemplary chemotherapeutic compounds for use in combinations provided herein include, but are not limited to, alkylating agents such as a platinum coordination complex, among other chemotherapeutic compounds provided herein. Exemplary platinum coordination complexes include, but are not limited to, cisplatin, carboplatin, oxaliplatin, DWA2114R, NK121, IS 3 295, and 254-S.

Provided herein are combinations of the viruses claimed and an anti-cancer agent, such as a cytokine, a chemokine, a growth factor, a photosensitizing agent, a toxin, an anti-cancer antibiotic, a chemotherapeutic compound, a radionuclide, an angiogenesis inhibitor, a signaling modulator, an anti-metabolite, an anti-cancer vaccine, an anti-cancer oligopeptide, a mitosis inhibitor protein, an antimitotic oligopeptide, an anti-cancer antibody, an anti-cancer antibiotic, an immunotherapeutic agent, hyperthermia or hyperthermia therapy or a bacterium. Provided herein are combinations of the viruses claimed and an anti-cancer agent, such as cisplatin, carboplatin, gemcitabine, irinotecan, an anti-EGFR antibody and an anti-VEGF antibody.

Provided herein are combinations as claimed where the compound and virus are formulated separately in two compositions. Provided herein are combinations as claimed where the compound and virus are formulated as a single composition.

Provided herein are the viruses as claimed herein for use in the treatment of a tumor, cancer or metastasis. Also provided herein are uses of the viruses claimed herein for preparation of a pharmaceutical composition for the treatment of a tumor, cancer or metastasis.

Provided herein are kits that contain a pharmaceutical composition or combination claimed herein and optionally instructions for administration thereof for treatment of cancer.

Provided herein are vaccines, such as a smallpox vaccine, containing a recombinant vaccinia virus claimed herein. Further, provided herein is a recombinant vaccinia virus claimed herein for administration as a vaccine, such as a smallpox vaccine, to a subject for generation of an immune response.

Disclosed herein in some embodiments, is a method of sensitizing a tumor to subsequent treatment modalities. The sensitization portion of the technology according to some embodiments may be performed using any of the approaches described herein.

In some embodiments, a subsequent treatment modality is selected from the group consisting of: radiation therapy, chemotherapy, immunotherapy, phototherapy, or a combination thereof. In some embodiments, sensitizing the tumor comprises administering irradiation to the subject. In some embodiments, the irradiation is ionizing radiation. In one embodiment, the sensitization will be achieved with local tumor irradiation, e.g. high-dose hypofractionation radiation therapy (HDHRT).

Ionizing radiation has a significant potential to modify the tumor microenvironment and facilitate immune-mediated tumor rejection. Specifically, radiation can induce remodeling of the abnormal tumor vessels and up-regulation of vascular cell adhesion molecules (e.g. VCAM-1) and chemokine secretion (e.g. CXCL16), resulting in efficient T-cell infiltration into the tumor. Other important effects of radiation include up-regulation of MHC class-I molecules, NKG2D ligands, and Fas/CD95, thus augmenting T-cell binding to and killing of the cancer cells. However, despite these significant pro-immunogenic effects, radiation by itself is insufficient to induce long-lasting and powerful enough anti-tumor immune responses leading to tumor eradication.

Radiation therapy includes, but is not limited to, photodynamic therapy, radionuclides, radioimmunotherapy and proton beam treatment.

In some embodiments, the subsequent treatment modality comprises administration of a chemotherapeutic compound. Chemotherapeutic compounds include, but are not limited to platinum; platinum analogs (e.g., platinum coordination complexes) such as cisplatin, carboplatin, oxaliplatin, DWA2114R, NK121, IS 3 295, and 254-S; anthracenediones; vinblastine; alkylating agents such as thiotepa and cyclosphosphamide; alkyl sulfonates such as busulfan, improsulfan and piposulfan; aziridines such as benzodopa, carboquone, meturedopa and uredopa; ethylenimines and methylamelamines including altretamine, triethylenemelamine, trietylenephosphoramide, triethylenethiophosphaoramide and trimethylolomelamime nitrogen mustards such as chiorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; substituted ureas; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; anti-cancer polysaccharides; polysaccharide-K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; cytosine arabinoside; cyclophosphamide; thiotepa; taxoids, such as paclitaxel and doxetaxel; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; XELODA; ibandronate; CPT11; topoisomerase inhibitor RFS 2000; difluoromethylomithine (DMFO); retinoic acid; esperamicins; capecitabine; methylhydrazine derivatives; Erlotinib (TARCEVA); sunitinib malate (SUTENT); and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone and toremifene (FARESTON); adrenocortical suppressants; and antiandrogens such as flutamide, nilutamide, bicalutamide, leuprolide and goserelin; and pharmaceutically acceptable salts, acids or derivatives of any of the above. Such chemotherapeutic compounds that can be used herein include compounds whose toxicities preclude use of the compound in general systemic chemotherapeutic methods.

In some embodiments, the subsequent treatment modality is selected from: local tumor irradiation, cytokine injections, antibody injections, and injections of stem cells secreting cytokines and/or chemokines.

### Administration of Treatment Modalities

The effective dosage of each of the treatment modalities disclosed herein, including the recombinant virus disclosed herein, employed in the combination therapy of the invention may vary depending on the particular treatment, compound or pharmaceutical composition employed, the mode of administration, the condition being treated, and the severity of the condition being treated. Thus, the dosage regimen of a treatment according to the invention is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

Methods of preparing pharmaceutical compositions comprising the relevant treatments disclosed herein are known in the art and will be apparent from the art, from known standard references, such as Remington's Pharmaceutical Sciences, Mack Publishing Company, Easton, Pa., 18th edition (1990).

### EXAMPLES

### Example 1—Materials and Methods

### Cell Lines

African green monkey kidney fibroblasts CV-1, human lung carcinoma A549, hypopharyngeal carcinoma cell line FaDu, and human prostate cancer cell line DU145 were obtained from the American Type Culture Collection. CV-1 cells were cultured in DMEM; A549 cells were cultured in RPMI 1640; and FaDu and DU145 were cultured in Eagle's minimal essential medium. All media were supplemented with 10% fetal bovine serum (Cellgro).

### Construction of Plasmids and Expression of Antibodies

The parental triple-mutant VACV GLV-1h68 was constructed as described previously (Zhang et al., Cancer Res. 67:10038-10046). Briefly, it contains three foreign gene-expression cassettes encoding Renilla luciferase-Aequorea GFP fusion protein (RUC-GFP), β-galactosidase, and β-glucuronidase integrated into the F14.5L, J2R, and A56R loci of the LIVP viral genome, respectively. The sequence of GLAF-5 was designed as described previously (Frentzen et al., Proc. Natl. Acad. Sci. USA 106: 12915-12920), and was synthesized by GENEART-Invitrogen. The DYKDDDDK tag (gac tac aag gat gac gac gac aag) was added into the C-terminal coding region of anti-EGFRVHH, resulting in anti-EGFRVHHFLAG. The DNA fragments were cloned into plasmids pCR-TK-P_SEL into the Sal I and Pac I sites, resulting in the plasmids pCR-TK-P_SEL (GLAF-5) and pCR-TK-P_SEL (anti-EGFRVHHFLAG), which were used for homologous recombination into the J2R locus in GLV-1h68 through double-reciprocal crossover, resulting in GLV-1h282 and GLV-1h442, respectively. GLV-1h446 and GLV-1h444 were generated similarly using the same plasmids and GLV-1h164 as the parental virus. The recombinant VACVs were sequence-confirmed. All VACVs were propagated in CV-1 cells and purified through sucrose gradients by a standard protocol.

For the detection of expression of antibodies, the cell samples were harvested at 24 hours after infection with each VACV strain at an MOI of 1, separated by 12% SDS-PAGE. The following antibodies were used: anti-DDDDK antibody (Abcam) for the detection of FLAG tag, the custom-made antibody G6 for the detection of scAbs, and anti-A27 antibody (GenScript Corporation) for the detection of the membrane protein of VACV.

The concentrations of the antibodies in mice sera were determined in standard ELISA assays using the commercial recombinant human EGFR (Abnova), FAP (R&D system), and VEGF (Sigma), as previously described (Frentzen et al., cited above).

### Viral Replication Assay

The cells were infected with VACVs at an MOI of 0.01 and the samples were harvested in triplicate at each time point after infection.

### Animal Studies

Five-to six-week-old nude male mice (Hsd:athymic Nude-Foxn1nu mice) were purchased from Harlan (Livermore, Calif.) and were cared for and maintained under the protocol approved by the Institutional Animal Care and Use Committee of Explora Biolabs (San Diego Science Center, San Diego, Calif.). 1×10⁶ of FaDu, 5×10⁶ of A549, or 1×10⁷ of DU145 cells were subcutaneously implanted into the right hind leg of the mice. The tumor volumes were measured weekly in three dimensions using a digital caliper and were calculated as ((length×width×(height−5))/2), and body weights were measured weekly. The net body weight for each animal was calculated by subtracting the total body weight at each time point from the weight of the tumor (assuming a tumor density of 1 g/cc). The percent change in net body weight was the difference between the net body weight of each animal at a specific time point and its net body weight immediately prior to treatment divided by the net body weight immediately prior to treatment, expressed as a percentage. A single dose of 2×10⁶ pfu/mouse (unless otherwise specified) in 100-μl PBS of each VACV strain was administered by retro-orbital injection when the tumor volume reached ˜450 mm³. One hundred microliters of PBS was injected as a negative control.

For treatment with therapeutic antibodies, mice were intraperitoneally (i.p.) injected with Avastin (5 mg/kg) and/or Erbitux (3 mg/kg) twice weekly for 5 weeks starting at 10 dpi.

The tumors and organs were excised and homogenized using a MagNA Lyser (Roche Diagnostics). The viral titers were determined in CV-1 cells by standard plaque assays.

### Histological Analysis

FaDu tumors were excised and paraffin-embedded, followed by the standard dehydration process. The tumor samples were cut into 5-μm sections and stained with hematoxylin and eosin (H&E). The sections were dewaxed and antigen retrieval was performed in a sodium citrate buffer. The following antibodies were used: anti-FAP (Abcam) and anti-A27L (GenScript Corporation). Biotinylated secondary antibodies (goat anti-rabbit; Jackson ImmunoResearch Laboratories) were used and detection was performed with Vectorstain Elite ABC reagent and Vector ImmPact DAB Peroxidase substrate (Vector Laboratories).

DU145 tumors were excised at 36 dpi, followed by paraformaldehyde fixation, and then cut into 100-μm sections. The blood vessels and cell proliferation were detected with anti-CD31 antibody (BD Pharmingen) and anti-Ki67 antibody (BD Pharmingen), respectively.

The examination of tumor sections was conducted with an MZ16 FA fluorescence stereomicroscope (Leica) equipped with a digital charge-coupled device camera (Leica). Digital images (1,300 to 1,030-pixel images) were processed using Adobe Photoshop 7.0 software.

### Statistical Analysis

The statistical significance of differences between groups of animals was analyzed using a two-tailed unpaired Student's t-test (Excel 2003; Microsoft, Redmond, Wash.) or one-way analysis of variance (ANOVA) in STATISTICS. A P-value<0.05 was considered significant.

### Example 2—Construction of Recombinant VACVs Encoding Individual Antibodies Targeting EGFR and FAP

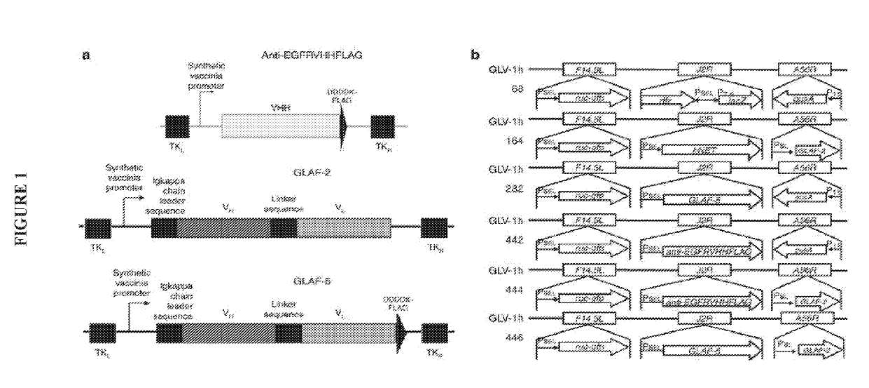

It has been previously shown that an anti-VEGF scAb (GLAF-1 or GLAF-2) expressed by VACVs (GLV-1h108 (ref 29) and GLV-1h164 (Buckel et al., Int. J. Cancer 133:2989-2999) significantly reduced tumor growth in several human tumor xenograft models and exhibited “Avastin-like mode of action” through the inhibitory effects on vascularity in the TME. EGFR and FAP are other important factors in the TME that are involved in the regulation of tumor initiation and development. To evaluate the effect of antibodies targeting EGFR and FAP on the therapeutic efficacy of oncolytic virotherapy, two new recombinant VACVs were constructed by replacing the lacZ expression cassette at the J2R locus of GLV-1h68 with an anti-EGFR nanobody (anti-EGFRVHHFLAG) expression cassette or with an anti-FAP scAb (GLAF-5) expression cassette, both under the control of the VACV synthetic early/late (PSEL) promoter, resulting in GLV-1h442 and GLV-1h282, respectively (FIG. 1a,b).

### Example 3—Virally Expressed Antibodies Targeting VEGF, EGFR, and FAP Significantly Enhance Virotherapy

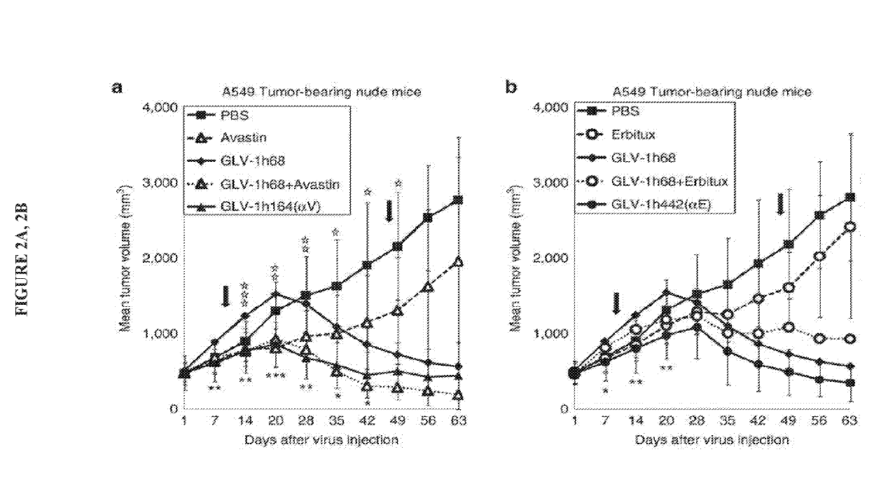

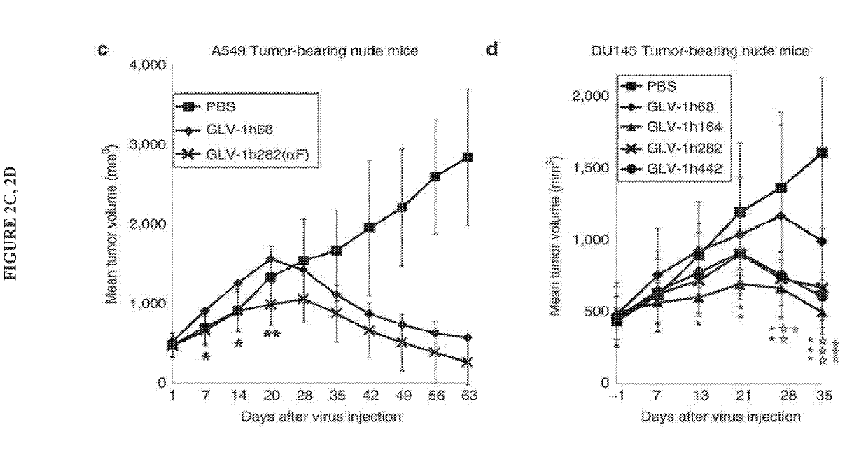

The antitumor effect of treatment with VACV strains encoding anti-VEGF, anti-FAP scAbs, or anti-EGFR nanobody was investigated in mice. The VACV strains or PBS (phosphate-buffered saline) was injected retro-orbitally at a single dose of 2×10⁶ plaque-forming units (PFU)/mouse into mice bearing different human tumor xenografts. Avastin or Erbitux was administered to mice twice weekly by intraperitoneal (i.p.) injection for a period of 5 weeks beginning 10 days after virus injection (as indicated by arrows in FIG. 2a-c). In the A549 xenograft model (n≧7) (FIG. 2a,b), PBS-treated tumors showed continuous growth until mice had to be sacrificed due to excessive tumor burden. The typical three-phase growth pattern of tumors in mice treated with GLV-h168 was observed (Zhang et al., cited above). The tumor volume exceeded the PBS-treated group at the beginning, followed by significant tumor growth arrest and then continuous tumor shrinkage. Mice treated with Avastin alone exhibited a reduction in tumor volume compared with PBS, but tumor growth was continuous (FIG. 2a). The treatment with GLV-1h68 in combination with Avastin yielded improved efficacy over either treatment alone whereas the therapeutic efficacy of GLV-1h164, expressing anti-VEGF scAb, was comparable to that of GLV-1h68 in combination with Avastin. The treatment with GLV-1h68 in combination with Erbitux yielded smaller tumors than the treatment with GLV-1h68 alone during the period of Erbitux administration, but tumor volume rebounded transiently after the treatment with Erbitux was ceased (FIG. 2b). In contrast, tumor growth in mice treated with GLV-1h442, expressing anti-EGFR nanobody, was consistently slower than in mice treated with GLV-1h68, and no rebound in tumor volume was observed. Additionally, treatment of mice with GLV-1h282, expressing anti-FAP scAb, also exhibited significantly smaller tumor volume than treatment with GLV-1h68 (FIG. 2c). Thus, an enhanced therapeutic effect on A549 tumor growth was observed on treatment of mice with GLV-1h164, GLV-1h442, or GLV-1h282, each expressing therapeutic antibodies, as compared with GLV-1h68. Moreover, the effect was superior to treatment with the therapeutic antibody alone and was either comparable or superior to the combination treatment of therapeutic antibody and GLV-1h68. A similar therapeutic effect was also observed in mice bearing DU145 tumor xenografts (n=5) (FIG. 2d).

### Example 4—Influences of Intratumorally Expressed Antibodies Targeting VEGF, EGFR. And FAP on the TME

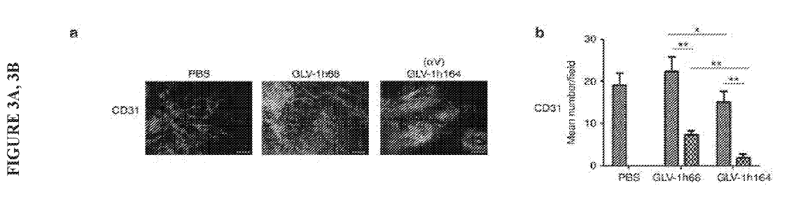

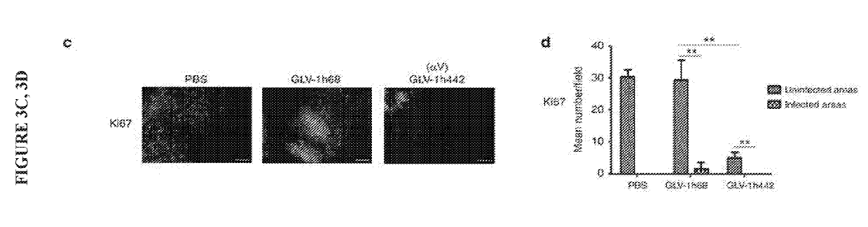

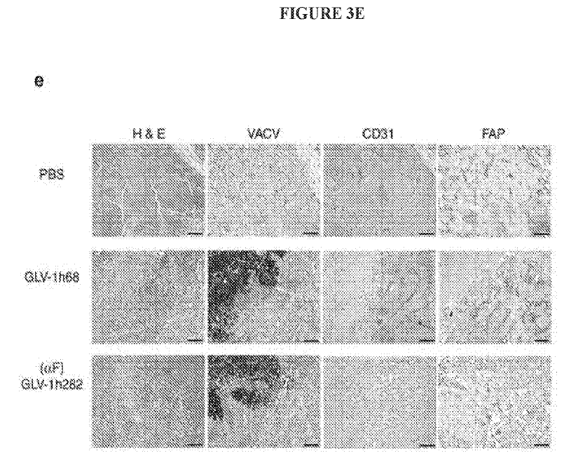

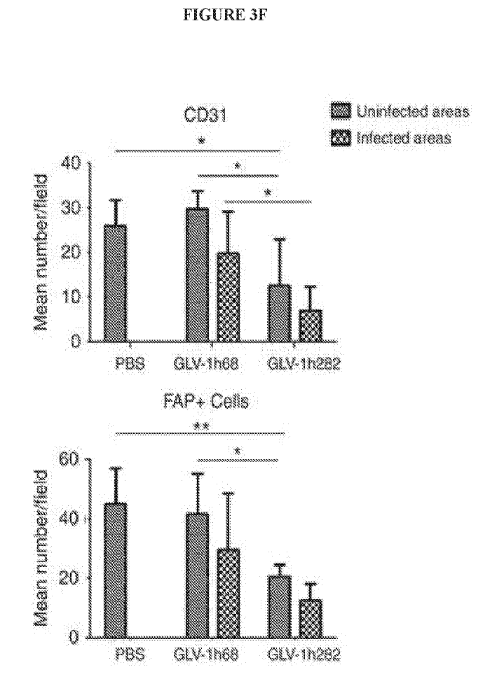

The effect of virally expressed anti-VEGF scAb on tumor vasculature was evaluated in DU145 tumors excised on 36-day post injection (dpi) of VACV. Immunohistochemistry (IHC) staining of tumor sections was performed to assess blood vessel density (BVD), determined by counting CD31+ blood vessels within tumor sections. The VACV infection was indicated by the fluorescence of virally expressed GFP (FIG. 3a). The VACV colonization resulted in a dramatic reduction in BVD in the infected areas of tumors compared with both PBS-treated tumors and uninfected areas of the same tumors (FIG. 3b). This was true for both GLV-1h68- and GLV-1h164-treated tumors. However, BVD in the uninfected areas of GLV-1h68-treated tumors was not significantly different from that in PBS-treated tumors. In contrast, treatment with GLV-1h164 significantly reduced BVD in the infected and uninfected areas of tumors compared with GLV-1h68-treated tumors. Thus, while VACV infection alone by GLV-1h68 reduced BVD in tumors, the effect was localized to the site of infection, whereas, the combination of VACV infection and expression of anti-VEGF scAb by GLV-1h164 not only further reduced BVD in the infected areas of tumors but also extended the effect to uninfected areas.

It is well known that overexpression of EGFR leads to uncontrolled cell growth. IHC staining with anti-Ki67 was performed to assess whether virally expressed anti-EGFR nanobody suppressed cell proliferation in tumors. As expected, colonization of tumors with either GLV-1h68 or GLV-1h442, expressing anti-EGFR nanobody, greatly reduced the number of Ki67+ cells in the infected areas compared with PBS-treated tumors (FIG. 3c). Interestingly, the number of Ki67+ cells in the uninfected areas of GLV-1h442-treated tumors was also significantly reduced compared with the uninfected areas of GLV-1h68-treated tumors (FIG. 3d). This suggested that the anti-EGFR nanobody secreted from cells infected with GLV-1h442 also acted on uninfected cells, reducing their proliferation.

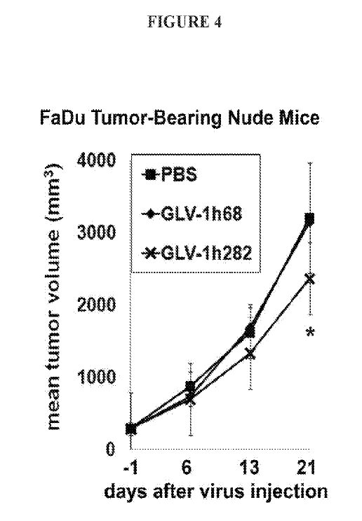

FAP is a mesenchymal stem cell (MSC) marker involved in angiogenesis. The effect of intratumorally expressed anti-FAP scAb was evaluated by counting the FAP+ cells and CD31+ cells in FaDu tumors, which express high levels of FAP. Although FaDu tumors did not respond to treatment with GLV-1h68, their growth was significantly inhibited by GLV-1h282 expressing anti-FAP scAb (FIG. 4). In GLV-1h68-colonized tumors, the numbers of CD31+ cells and FAP+ cells were greatly reduced in the infected areas, whereas no effect was observed in the uninfected areas compared with PBS-treated tumors (FIG. 3e,f). In contrast, the numbers of CD31+ cells and FAP+ cells in GLV-1h282-treated tumors were significantly reduced in both the infected and uninfected areas compared with GLV-1h68-treated tumors.

### Example 5—Construction of Additional New Recombinant VACVs Expressing Two Antibodies Targeting VEGF and EGFR or VEGF and FAP

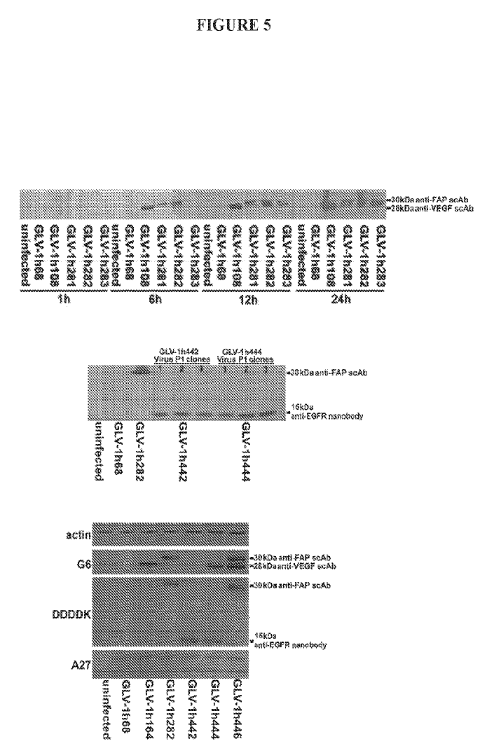

Based on the positive effects of treatment with VACVs expressing single antibodies, additional new recombinant VACVs expressing two antibodies targeting VEGF and EGFR or VEGF and FAP were constructed. The expression cassette for anti-EGFR nanobody (anti-EGFRVHHFLAG) or anti-FAP scAb (GLAF-5) was inserted into the J2R locus of GLV-1h164, which also contained the anti-VEGF (GLAF-2) expression cassette, to replace the human norepinephrine transporter (hNET) expression cassette, resulting in GLV-1h444 and GLV-1h446, respectively (FIG. 1b). To verify the expression of each antibody from the new recombinant VACVs, A549 cells were infected with the new VACV strains and cell lysates were analyzed by western blot. The results showed that the respective antibodies were expressed from each virus as intended (FIG. 5).

### Example 6—Virally Expressed Two Antibodies do not Show Negative Effects on Viral Replication Efficiency

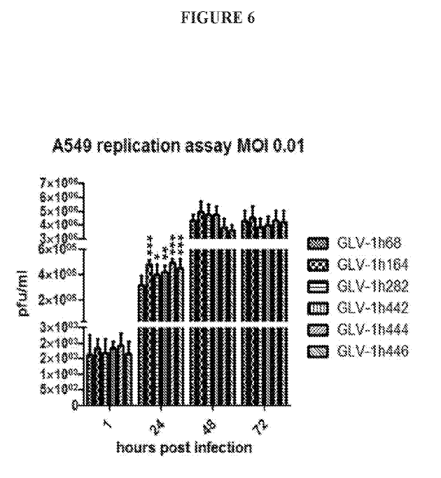

Viral replication assays were performed in A549 cells at a multiplicity of infection (MOI) of 0.01 (FIG. 6). The recombinant VACVs expressing single or two antibodies showed significantly higher replication efficiency than GLV-1h68 at 24-hour post-infection (hpi). However, there was no significant difference in the replication efficiency at 48 or 72 hpi. Similar results were obtained in DU145 cells. Thus, the expression of two antibodies in VACVs did not show negative effects on their overall replication efficiency in cell culture.

### Example 7—Virally Expressed Two Antibodies Targeting the TME Further Improve Virotherapy

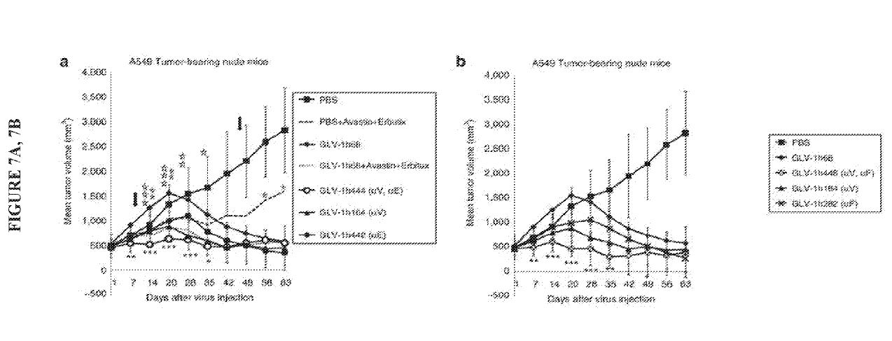

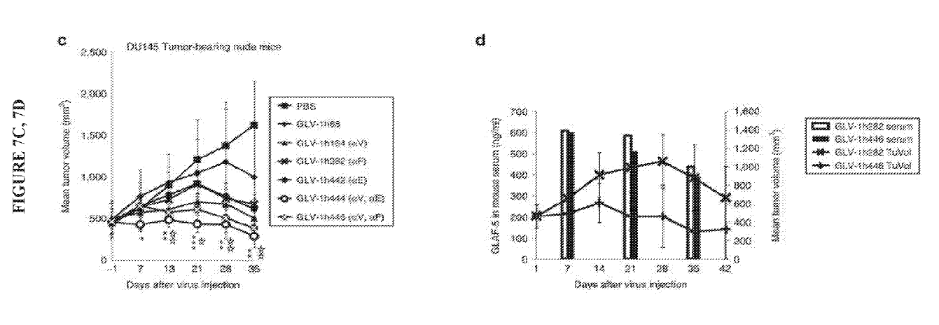

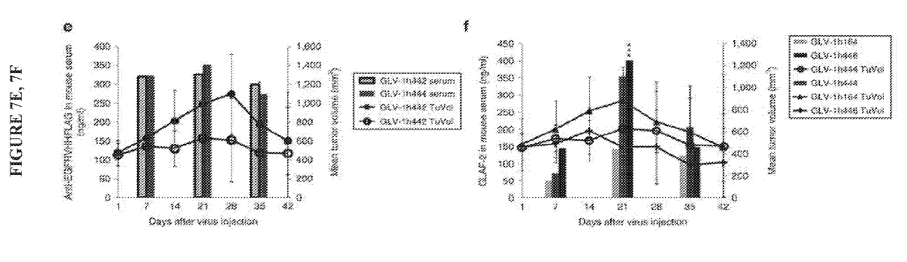

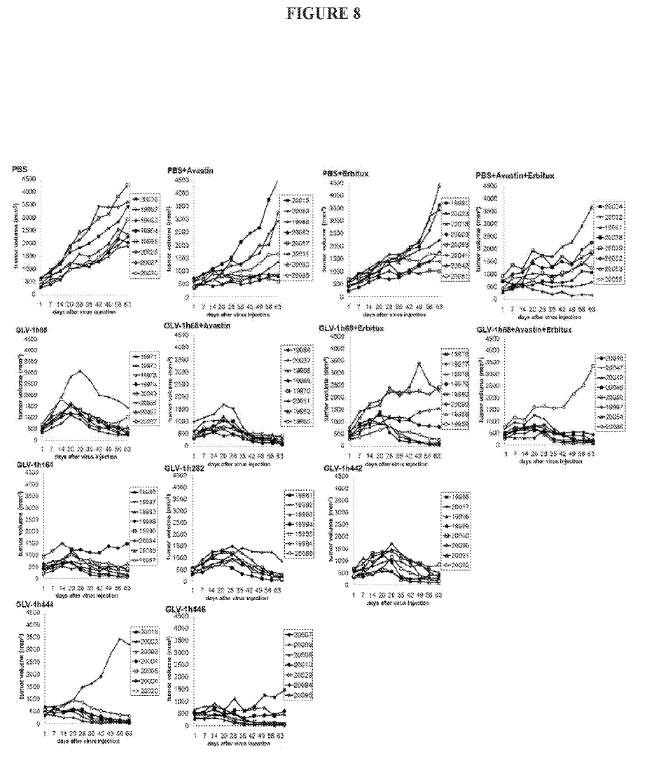

Encouraged by the results from the recombinant VACVs expressing single antibodies targeting the TME, an evaluation was performed concerning whether two antibodies expressed from the same VACV would further improve virotherapy. A single dose of each VACV strain was injected into mice bearing A549 tumor xenografts (FIG. 7a,b). Control animals were treated with PBS or the combination of Avastin and Erbitux. The treatment with Avastin and Erbitux twice weekly for 5 weeks beginning at 10 dpi delayed tumor growth during the period of antibody treatment, but tumor growth resumed after the termination of antibody treatment. Surprisingly, tumors in mice treated with GLV-1h444 or GLV-1h446, both expressing two antibodies, showed only minimal growth over the period of the experiment. In contrast, tumors in mice treated with VACVs expressing single antibodies initially grew as fast as or only slightly slower than tumors treated with PBS before tumor growth slowed significantly, followed by tumor shrinkage. Overall, the tumor volumes in mice treated with GLV-1h444 or GLV-1h446, expressing two antibodies, were less than the tumor volumes in mice treated with VACVs expressing single antibodies. The tumor growth curves of individual mice bearing A549 tumors are shown in FIG. 8. Similar patterns of tumor growth were obtained with DU145 tumors (FIG. 7c).

### Example 8—Virally Expressed Antibodies are Detectable in Sera of Treated Mice

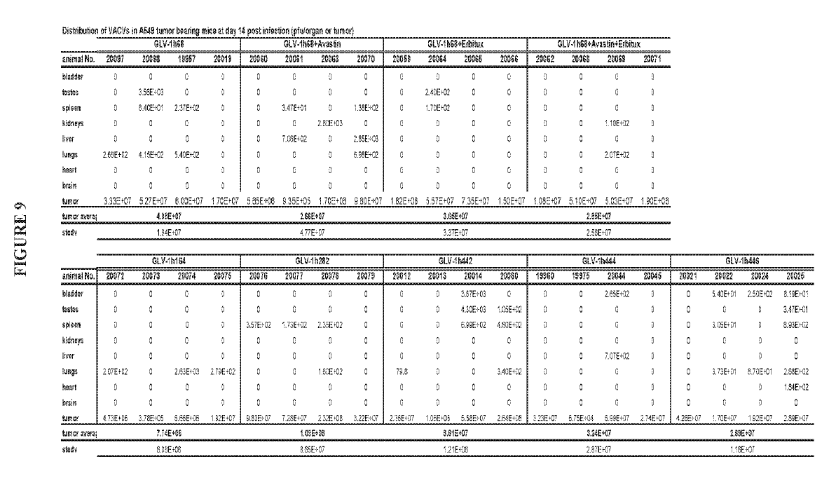

After the verification of antibody expression in virus-infected cells in culture, the presence of antibodies in tumor-bearing mice was also investigated. The blood samples were collected retro-orbitally from the same mice at 7, 21, and 35 dpi. All samples were tested for the presence of anti-FAP scAb with FAP-precoated plates, anti-EGFR nanobody with EGFR-precoated plates, and anti-VEGF scAb with VEGF-precoated plates. All of the antibodies were detectable at all three time points (FIG. 7d-f). The expression of GLAF-2 was lower in mice treated with GLV-1h164 (single antibody) than in mice treated with GLV-1h444 and GLV-1h446 (two antibodies) at 7 and 21 dpi, consistent with the lower viral titers in tumors at 14 dpi in the GLV-1h164-treated group (FIG. 9). Nonetheless, in all cases, the expression of antibodies at the early stages (day 7 and 21) coincided with low tumor volumes and at the later stage (day 35) preceded tumor shrinkage.

### Example 9—Virally Expressed Two Antibodies do not Alter Viral Distribution and Toxicity in Mice

The possible adverse effect of antibody-expressing VACV administration in mice was evaluated by assessing the change in net body weight over the course of treatment. In both A549 and DU145 tumor xenograft models, no significant change in the mean net body weight was observed for any of the treated or control groups (FIG. 10a-c). Also evaluated was the viral bio-distribution in different organs and tumors in A549 tumor-bearing mice at 14 dpi as determined by standard viral plaque assays (FIG. 9). Significant viral titers were detected in all treated tumors and either no titers or minimal titers were detected in other organs. The viral titers in tumors treated with GLV-1h282, expressing anti-FAP scAb, and GLV-1h442, expressing anti-EGFR nanobody, were higher than in tumors treated with GLV-1h68, although not statistically significant, whereas the viral titer of GLV-1h164, expressing anti-VEGF scAb, in tumors was significantly lower than the other virus strains (GLV-1h164 versus GLV-1h68, P=0.02, and versus GLV-1h446, P=0.04).

### Example 10—Effects of Intratumorally Expressed Two Antibodies Targeting VEGF and EGFR or VEGF and FAP on the TME

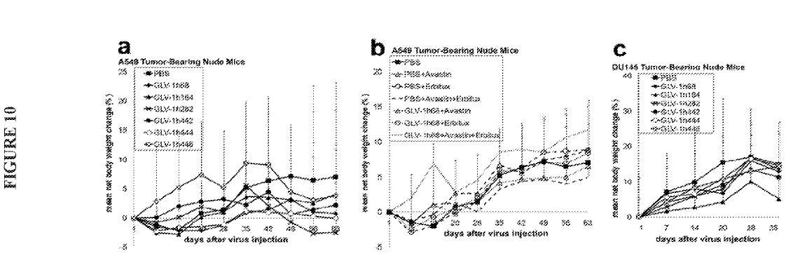

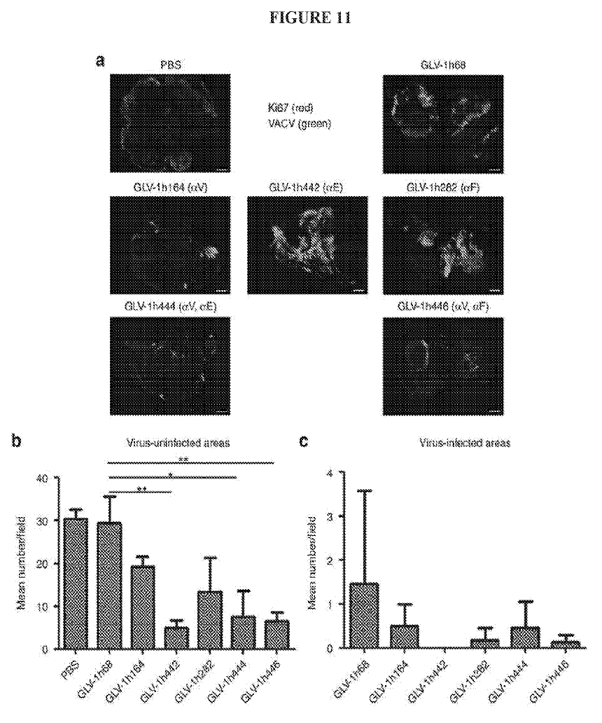

The influence of two virally expressed antibodies on the TME was evaluated by examining cell proliferation and BVD in DU145 tumor xenografts by staining tumor sections obtained at 36 dpi with anti-Ki67 and anti-CD31 antibodies. The VACV infection was confirmed by GFP fluorescence. The representative images of IHC staining of proliferating Ki67+ cells are shown in FIG. 11a. In the infected areas of the tumors, Ki67+ cells were significantly reduced after treatment with any of the VACV strains, including GLV-1h68 (FIG. 11c). In the uninfected areas of VACV-treated tumors, Ki67+ cells were significantly reduced only after treatment with the single anti-EGFR nanobody-expressing VACV, GLV-1h442, and the two antibody-expressing VACVs, GLV-1h444 and GLV-1h446 (FIG. 11b).

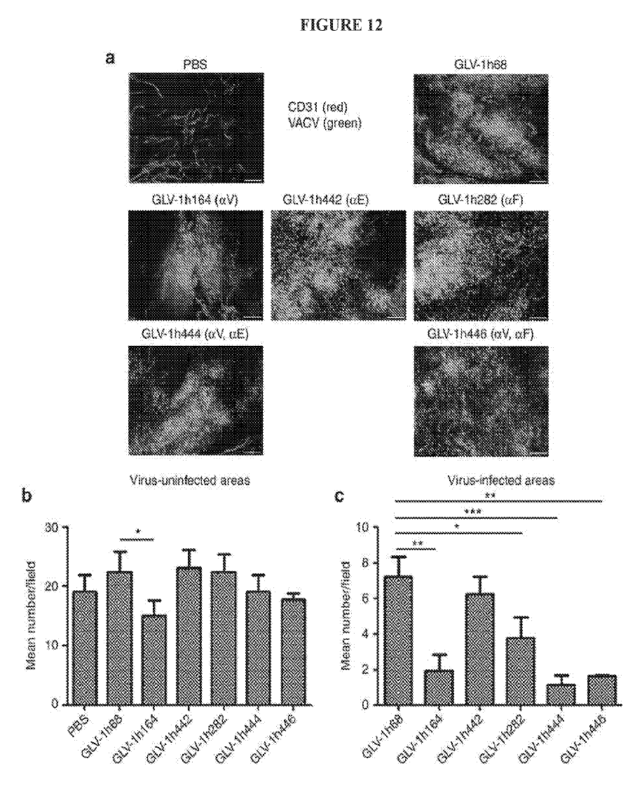

The images of DU145 tumor sections stained with anti-CD31 antibody showed that the infected areas of tumors were significantly reduced in BVD compared with the uninfected areas regardless of the VACV used for treatment (FIG. 12a-c). Compared with GLV-1h68, a significant further reduction in BVD was observed with VACVs expressing anti-VEGF antibody (GLAF-2), either singly or in combination with the other antibody. As expected, BVD in tumors treated with GLV-1h442, expressing anti-EGFR but not anti-VEGF, was not significantly different than that in tumors treated with GLV-1h68, but the infected areas treated with GLV-1h444 and GLV-1h446 showed a further reduced BVD.

All publications cited herein are incorporated by reference.

The disclosures illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Other embodiments are set forth within the following claims.

1. A method for treating a solid tumor in a subject, comprising administering to the subject a recombinant virus that can infect a cell in the tumor, or a cell comprising a virus that can infect a cell in the tumor, wherein the virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein targeting the TME.

2. The method of claim 1, wherein the virus is an oncolytic virus.

3. The method of claim 2, wherein the oncolytic virus is a vaccinia virus.

4. The method of claim 3, wherein the virus is a replication-competent oncolytic vaccinia virus (VACV).

5. The method of claim 1, wherein the virus expresses two or more antibodies that target the TME.

6. The method of claim 5, where the two or more antibodies are selected from: (a) an antibody that binds to a protein that stimulates angiogenesis and/or vascularization, (b) an antibody that binds to a receptor tyrosine kinase selected from epidermal growth factor receptor (EGFR), Her2/c-neu, Her3 and Her4, and (c) an antibody that binds to a protein involved in the development of epithelial-mesenchymal interactions.

7. The method of claim 5, wherein the two or more antibodies are selected from: an antibody that binds to vascular endothelial growth factor (VEGF), an antibody that binds to epidermal growth factor receptor (EGFR), and an antibody that binds to fibroblast activation protein (FAP).

8-9. (canceled)

10. The method of claim 7, wherein the antibody that binds to VEGF is G6-31, and/or the antibody that binds to EGFR is anti-EGFRVHH, and/or the antibody that binds to FAP is M036.

11-17. (canceled)

18. The method of claim 4, wherein the VACV is selected from GLV-1h444 and GLV-1h446.

19. The method of claim 1, further comprising administering an additional cancer therapy.

20. (canceled)

21. The method of claim 1, wherein the tumor is selected from: glioblastoma, breast carcinoma, lung carcinoma, prostate carcinoma, colon carcinoma, ovarian carcinoma, neuroblastoma, central nervous system tumor, and melanoma.

22. The method of claim 1, wherein the virus is intravenously delivered to the subject.

23. The method of claim 1, wherein the virus is intravenously delivered directly into a tumor, or delivered to the subject within the region of a tumor.

24. The method of claim 1, further comprising providing to the subject at least one additional virus that can infect a cell in the tumor, wherein the at least one additional virus expresses one or more of: (i) an antibody that targets the tumor microenvironment (TME) or (ii) a stimulatory or inhibitory protein targeting the TME, wherein the one or more: (i) an antibody that targets the TME or (ii) a stimulatory or inhibitory protein targeting the TME expressed by the at least one additional virus are different compared to the antibodies expressed by the virus provided according to claim 1.

25. The method of claim 1, further comprising administering to the subject at least one additional virus, wherein the at least one additional virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein that targets the TME, wherein the at least one additional virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein that targets the TME that are different from those expressed by the virus provided according to claim 1.

26. A recombinant virus that can infect a cell in a solid tumor, wherein the virus expresses: (i) two or more antibodies that target the tumor microenvironment (TME); (ii) two or more of a stimulatory or inhibitory protein targeting the TME, or (iii) at least one antibody that targets the TME and at least one of a stimulatory or inhibitory protein that targets the TME.

27-30. (canceled)

31. The recombinant virus of claim 26, wherein the virus expresses two or more antibodies selected from: (i) an antibody that binds to a protein that stimulates angiogenesis and/or vascularization, (ii) an antibody that binds to a receptor tyrosine kinase selected from epidermal growth factor receptor (EGFR), Her2/c-neu, Her3 and Her4, and (iii) an antibody that binds to a protein involved in the development of epithelial-mesenchymal interactions.

32-51. (canceled)

52. A host cell comprising a recombinant virus of claim 26.

53. A tumor cell comprising a recombinant virus of claim 26.

54. A mammalian organism comprising, or infected by, the recombinant virus of claim 26.

55-58. (canceled)
